(19) United States
(12) Patent Application Publication
(10) Pub. No.: US 2011/0045530 A1
(43) Pub. Date: Feb. 24, 2011

(54) ENZYMATIC CONJUGATION OF BIOACTIVE MOIETIES

(76) Inventors: Peter Jan Leonard Mario Quaedflieg, Elsloo (NL); Bartholomeus Johannes Margretha Plum, Ulestraten (NL); Aylvin Jorge Angelo Athanasius Dias, Maastricht (NL); Bas Ritzen, Nijmegen (NL); Claudia Cusan, Aachen (DE); Catharina Hubertina Maria Schepers, Stein (NL)

Correspondence Address: NIXON & VANDERHYE, PC, 901 NORTH GLEBE ROAD, 11TH FLOOR, ARLINGTON, VA, 22203, US

(21) Appl. No.: 12/867,320

(22) PCT Filed: Feb. 13, 2009
(86) PCT No.: PCT/EP09/51714

§ 371 Date: Nov. 1, 2010

(30) Foreign Application Priority Data
Feb. 13, 2008 (EP) 08002627.1

(51) Int. Cl. C12P 21/00; C12P 13/04

(52) U.S. Cl. 435/68.1; 435/106

(57) ABSTRACT

The present invention relates to a method for selective conjugation of bioactive moieties to a polymer or polymerisable compound. The method is more specifically related to the selective conjugation of bioactive moieties to a pendant carboxylic acid, ester or thioester group in which the pendant group is part of a polymer or a polymerisable compound, wherein the method comprises contacting the polymer or polymerisable compound with a hydrolytic enzyme to catalyse the conjugation between the bioactive moiety and the pendant carboxylic acid, ester or thioester group. The conjugation of the bioactive moieties may occur prior to, during or after polymerization of the polymerisable compound. The conjugation of the bioactive moieties may also occur after the polymer is given a form.

The invention relates to a method for selective conjugation of bioactive moieties to a polymer or polymerisable compound.

Polymers or polymerisable compounds, such as monomers, macromers or prepolymers, conjugated with bioactive moieties find wide-spread use in biomedical applications. For instance, the bioactive moiety can be conjugated via a functional group, e.g. a carboxylic acid, which is part of the polymer or polymerisable compound. However, it is often desirable or even necessary that the carboxylic acid group is protected at some stage in the preparation of the conjugated product, in order to allow a specific process step to take place efficiently and/or to avoid an undesired side reaction due to the presence of a free (i.e. unprotected) carboxylic acid group. Often, the carboxylic acid is protected by esterification with a hydrocarbon.

Before being able to chemically conjugate a bioactive moiety to the protected carboxylic acid, a deprotection step is needed. However, such deprotection may be troublesome, in particular in case the polymer or polymerisable compound comprises one or more other hydrolysable groups such as further ester or thioester groups in addition to the protected carboxylic acid group.

Hydrolysable groups such as ester or thioester groups are normally hydrolysed by an acid or base in an aqueous environment. It is however known that such a hydrolysis is not selective. In some cases a selective hydrolysis is required in particular if for example a polymer or polymerisable compound comprises one or more other hydrolysable groups for example multiple ester groups. It is for example known that a selective hydrolysis of a t-butyl ester over some other ester or thioester groups can be achieved preferentially in a chemical process for example with trifluoroacetic acid (TFA) in a dry organic solvent. However, several disadvantages are associated with this process. For an efficient deprotection of the ester it is generally required to use a large excess of TFA (>10 equivalents). The highly acidic conditions make this form of deprotection unsuitable for compounds that are not stable in strongly acidic conditions. The reaction is carried out in a dry solvent as a trace of water during the TFA-mediated deprotection would usually be sufficient to cause extensive hydrolysis of other hydrolysable groups, in particular other ester or thioester functions in the molecule. Complete or almost complete removal of TFA is laborious (and expensive) but of crucial importance, in particular in case a functional group, e.g. a functional group which is part of a bioactive moiety, is to be coupled to the carboxylic acid, since the presence of TFA in the coupling step may be detrimental to the conjugation reaction.

In case of chemical conjugation of a bioactive moiety to a polymer or polymerisable compound the bioactive moiety should be at least partially protected on reactive groups in order to avoid side reactions with the chemical coupling agent.

The chemical coupling agents are moreover expensive, not recyclable and environmentally unfriendly.

The use of the protected bioactive moiety moreover requires one or more further deprotection steps after the conjugation reaction which may be a challenge.

It is an object of the present invention to overcome one or more disadvantages such as indicated above.

It is a further object of the present invention to provide a new method to conjugate bioactive moieties efficiently to a polymer or polymerisable compound.

It is still a further object of the present invention to provide a method in which the deprotection step of carboxylic acid groups present in the polymer or polymerisable compound is not required.

It is still a further object of the present invention to provide a method which does not require expensive coupling reagents or multiple steps in the conjugation process.

It is a further object of the present invention to provide a method in which the bioactive moieties require less or no protective groups on their reactive functionalities before conjugation.

It has now been found possible to selectively conjugate bioactive moieties to a polymer or polymerisable compound.

Accordingly, the present invention relates to a method for the selective conjugation of bioactive moieties to a pendant carboxylic acid, ester or thioester group in which the pendant group is part of a polymer or a polymerisable compound, wherein the method comprises contacting the polymer or polymerisable compound with a hydrolytic enzyme to catalyse the conjugation between the bioactive moiety and the pendant carboxylic acid, ester or thioester group

It has surprisingly been found possible to conjugate a bioactive moiety to a pendant carboxylic acid, ester or thioester group present in a polymer or polymerisable compound with a high degree of selectivity over one or more other groups, for example other ester groups, thioester groups, urethane groups or urea groups which might be present in the backbone chain of the polymer or polymerisable compound.

An advantage of the method of the present invention is that the enzymatic process according to the present invention is environmentally friendly in comparison to a chemical conjugation process.

A further advantage is that bioactive moieties can be conjugated selectively to sterically large polymers or polymerisable compounds by a catalytic amount of a cheap and recyclable enzyme.

A still further advantage is that only partial or no protection of the reactive functionalities of the bioactive moiety is required before conjugation.

It is still a further advantage that the bioactive moiety can be conjugated selectively to protected as well as unprotected carboxylic acid groups whereby in case of protection no deprotection step is required, e.g. in the case of ester or thioester groups.

In case that the polymer or polymerisable compound has an optically active center to which the bioactive moiety is attached, it is a further advantage that no or less racemisation of the polymer or polymerisable compound takes place during the conjugation reaction.

As used herein, the term “polymer” denotes a structure that essentially comprises a multiple repetition of units derived, actually or conceptually, from molecules of low relative molecular mass. Such polymers may include crosslinked networks, dendrimeric and hyperbranched polymers and linear polymers. Oligomers are considered a species of polymers, i.e. polymers having a relatively low number of repetitions of units derived, actually or conceptually, from molecules of low relative molecular mass.

Polymers may have a molecular weight of 200 Da or more, 400 Da or more, 800 Da or more, 1000 Da or more, 2000 Da or more, 4000 Da or more, 8000 Da or more, 10 000 Da or more, 100 000 Da or more or 1 000 000 Da or more. Polymers having a relatively low mass, e.g. of 8000 Da or less, in particular 4000 Da or less, more in particular 1000 Da or less may be referred to as oligomers.

By a pendant carboxylic acid, ester or thioester is meant a carboxylic acid, ester or thioester group that is not in the polymer backbone or will not be in the resultant polymer backbone in a subsequent polymerisation step.

It is in particular surprising that the invention allows the selective conjugation of bioactive moieties with a pendant sterically difficult accessible carboxylic acid, ester or thioester group in a compound such as a polymer or oligomer or a large polymerisable compound, for example compounds comprising more than one polymerisable moiety.

The present invention in particular relates to a method wherein the pendant carboxylic acid, ester or thioester group is part of a polymer or a polymerisable compound comprising (a) at least two polymerisable moieties and (b) at least one amino acid residue.

The method according to the present invention is particularly useful to selectively conjugate a bioactive moiety with a pendant carboxylic acid, ester or thioester group of a polymer or polymerisable compound comprising (a) at least two polymerisable moieties, and (b) at least one amino acid residue of an amino acid comprising at least two amine groups of which at least two amine groups have formed a urea group, a thio-urea group, a urethane group or a thio-urethane group.

The invention thus allows the selective conjugation of a pendant carboxylic acid, ester or thioester group in a polymer or polymerisable compound which may be obtained from commercially readily available or easily synthesisable starting compounds. For example a urethane can be prepared from a diamino acid of which the carboxylic acid function is protected with a primary alkyl ester, for example a methylester, such as L-lysine methylester.

Further, it is advantageous that a highly selective conjugation with bioactive moieties is achievable without needing a stoichiometric amount of an expensive and environmentally unfriendly coupling agent.

The polymer or polymerisable compound may comprise, in addition to the pendant carboxylic acid, ester or thioester group, a moiety selected from urea groups, thio-urea groups, urethane groups, thio-urethane groups, other ester groups, amide groups, glycopeptide groups, carbonate groups, sulphones and carbohydrate groups.

The method according to the present invention is more in particular useful to selectively conjugate bioactive moieties to a polymer or polymerisable compound represented by the formula I wherein:

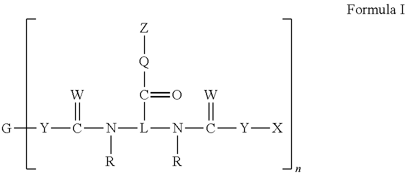

G is a residue of a polyfunctional compound having at least n functional groups or a moiety X.
X represents a moiety comprising a polymerisable group.
in case that G=X, formula I represents a polymerisable compound.
in case that G is different from X, formula I represents a polymer or oligomer.
each Y independently represents O, S or NR.
each W independently represents O or S.
Q represents O or S.
each R independently represents hydrogen or a group selected from substituted and unsubstituted hydrocarbons which optionally contain one or more heteroatoms.
L represents a substituted or unsubstituted hydrocarbon group which optionally contains one or more heteroatoms.
n is an integer having a value of at least 1 and
Z is H or a substituted or unsubstituted hydrocarbon group.

In principle, G is a multifunctional polymer or oligomer optionally functionalised with an —OH, —NH₂, —RNH or —SH, where the group that reacts to give formula I is —OH, a primary amine, a secondary amine or —SH. In case that G is not X, G may be selected from polyesters, polythioesters, polyorthoesters, polyamides, polythioethers and polyethers.

In particular, G may be selected from polylactic acid (PLA); polyglycolide (PGA); polyanhydrides; polytrimethylenecarbonates; polyorthoesters; polydioxanones; poly-ε-caprolactones (PCL); polyurethanes; polyvinyl alcohols (PVA); polyalkylene glycols, for example polyethyleneglycol (PEG); polyalkylene oxides, preferably selected from polyethylene oxides or polypropylene oxides; polyethers; poloxamines; polyhydroxy acids; polycarbonates; polyaminocarbonates; polyvinyl pyrrolidones; polyethyl oxazolines; carboxymethyl celluloses; hydroxyalkylated celluloses, such as hydroxyethyl cellulose and methylhydroxypropyl cellulose; and natural polymers, such as polypeptides, polysaccharides and carbohydrates, such as polysucrose, hyaluranic acid, dextran and derivatives thereof, heparan sulfate, chondroitin sulfate, heparin, alginate, and proteins such as gelatin, collagen, albumin, or ovalbumin; and co-oligomers, copolymers, and blends thereof comprising any of these moieties.

The moiety G may be chosen based upon its biostability and/or biodegradability properties. For providing a compound or polymer or article with a high biostability, polyethers, polythioethers, aromatic polyesters, aromatic thioesters are generally particularly suitable. Preferred examples of oligomers and polymers that impart biodegradability include aliphatic polyesters, aliphatic polythioesters, aliphatic polyamides and aliphatic polypeptides.

Preferably, G is selected from polyesters, polythioesters, polyorthoesters, polyamides, polythioethers and polyethers. Good results have in particular been achieved with polyethers, in particular with a polyalkylene glycol, more in particular with polyethyleneglycol (PEG).

For a hydrophobic polymer, G may suitably be selected from hydrophobic polyethers such as polybutylene oxide or polytetramethyleneglycol (PTGL).

A polyalkylene glycol, such as PEG, is advantageous in an application wherein a product may be in contact with a body fluid containing proteins, for instance blood, plasma, serum or an extracellular matrix. It may in particular show a low tendency to foul (low non-specific protein absorption) and/or have an advantageous effect on the adhesion of biological tissue. A low fouling is desirable when signaling peptides or biological molecules are required to communicate with cells. In this case it is important that the signaling peptides or biological molecules are not camouflaged or covered by non-specific protein adsorption.

The number average molecular weight (Mn) of the moiety G is usually at least 200 g/mol, in particular at least 500 g/mol. For an improved mechanical property, Mn preferably is at least 2000 g/mol. The number average molecular weight of the moiety G is usually up to 100 000 g/mol. The number average molecular weight is determinable by size exclusion chromatography (SEC).

The hydrocarbon group Z may in principle be any substituted or unsubstituted alkyl or aryl group, optionally comprising one or more heteroatoms, such as one or more heteroatoms selected from the group of N, S, O, CI, F, Br and I. Usually, the number of C atoms is 1-20, preferably 1-10, more preferably 1-6. The hydrocarbon may be linear, branched or cyclic. Most preferred are alkyl groups, because alkyl groups are highly suitable as a protective group. The alkyl group may be an unsubstituted alkyl group or a substituted alkyl group, for example a hydroxyalkyl group.

Preferably the alkyl group may be methyl, ethyl, or n-propyl. Most preferably the alkyl group is a methyl group.

In principle, the polymerisable moiety (such as “X”, in Formula I) in the polymerisable compound can be any moiety that allows formation of a polymer. In particular it may be chosen from moieties that are polymerisable by an addition reaction. Such type of reaction has been found easy and well-controllable. Further, the polymerization reaction may be carried out without formation of undesired side products, such as products formed from leaving groups.

Preferably, the polymerisable moiety allows radical polymerisation. This has been found advantageous as it allows initiating a polymerisation, in the presence of a photo-initiator, by electromagnetic radiation, such as UV, visible light, microwave, near-IR, gamma radiation, or by electron beam instead of thermally initiating the polymerisation reaction. This allows rapid polymerisation, with no or at least a reduced risk of thermal denaturation or degradation of (parts of) the polymer or polymerisable compound. Thermal polymerisation may be employed, in particular in case no biological moiety or moieties are present that would be affected by heat. E.g. heat-polymerisation may be employed when one or more oligo-peptides and/or proteins form or are part of the bioactive moiety of which the active sites are not affected by the high temperature required for polymerisation at elevated temperatures.

Preferred examples of the polymerisable moiety (“X”, in Formula I) include groups comprising an unsaturated carbon-carbon bond—such as a C═C bond (in particular a vinyl group) or a C≡C group (in particular an acetylene group), thiol groups, epoxides, oxetanes, hydroxyl groups, ethers, thioethers, HS—, H₂N—, —COOH, HS—(C═O)— or a combination thereof, in particular a combination of thiol and C═C groups.

In particular preferred is a polymerisable moiety selected from the group consisting of an acrylate including hydroxyl(meth)acrylates; alkyl(meth)acrylates, including hydroxyl alkyl(meth)acrylates; vinylethers; alkylethers; unsaturated diesters and unsaturated diacids or salts thereof (such as fumarates); and vinylsulphones, vinylphosphates, alkenes, unsaturated esters, fumarates, maleates or combinations thereof. More preferred is a moiety selected from acrylates, methacrylates, itaconates, vinylethers, propenylethers, alkylacrylates and alkylmethacrylates. Most preferred is a moiety selected from (meth)acrylates and alkyl(meth)acrylates, especially hydroxy alkylmethacrylates and hydroxy alkylacrylates. Such moiety can be introduced in the polymerisable compound of the invention starting from readily available starting materials and shows good biocompatibility, which makes them particularly useful for in vivo or other medical applications.

Good results have in particular been achieved with a polymerisable compound wherein the X-Y moiety represents hydroxyethylacrylate or hydroxyethylmethacrylate.

In a further preferred embodiment, the polymerisable moiety X is represented by the formula —R₁R₂C═CH₂, wherein R₁ is chosen from the group of substituted and unsubstituted, aliphatic, cycloaliphatic and aromatic hydrocarbon groups that optionally contain one or more moieties selected from the group consisting of ester moieties, ether moieties, thioester moieties, thioether moieties, urethane moieties, thiourethane moieties, amide moieties and other moieties comprising one or more heteroatoms, in particular one or more heteroatoms selected from S, O, P and N. R₁ may be linear or branched. In particular R₁ may comprise 1-20 carbon atoms, more in particular it may be a substituted or unsubstituted C₁ to C₂₀ alkylene; more in particular a substituted or unsubstituted C₂ to C₁₄ alkylene. R₂ is chosen from the group of hydrogen and substituted and unsubstituted alkyl groups, which alkyl groups optionally contain one or more heteroatoms, in particular one or more heteroatoms selected from P, S, O and N. R₂ may be linear or branched. In particular, R₂ may be hydrogen or a substituted or unsubstituted C₁ to C₆ alkyl, in particular a substituted or unsubstituted C₁ to C₃ alkyl.

The amino acid moiety (“L” in formula I) is a substituted or unsubstituted hydrocarbon, which may contain heteroatoms, such as N, S, P and/or O.

The amino acid moiety L may be based on a D-isomer or an L-isomer of an amino acid. Preferably, L is a C1-C20 hydrocarbon, more preferably, L is a linear or branched C1-C20 alkylene, even more preferably a C2-C12 alkylene, most preferably a C3-C8 alkylene, wherein the alkylene may be unsubstituted or substituted and/or optionally contains one or more heteroatoms. The number of carbon atoms is preferably relatively low, such as 8 or less.

In case the polymer or polymerisable compound is intended to be used in a medical application, more in particular in case it is intended to be used in vivo, it is preferred that the amino acid moiety is based upon a natural amino acid. This is in particular desired in case the compound or polymer is biodegradable. In view thereof, preferred amino acid moieties are moieties of lysine, hydroxylysine, methylated lysine, arginine, asparagine, diaminobutanoic acid and glutamine in the L- or D-configuration or as a racemate or as any mixture of D or L-isomers. Preferably the amino acid moieties are in the L-configuration. Good results have in particular been achieved with L-lysine.

More in particular the present invention relates to a method wherein the polymerisable compound is represented by formula I in which
G is X;
each Y is O and each X represents a moiety comprising hydroxyalkylene, hydroxyethylacrylate or hydroxyethylmethacrylate,
each R represents hydrogen,
L represents an amino acid moiety,
n=1,
W is O,
Q is O,
Z is H or an alkyl group with 1-6 C atoms.

Still more preferably the present invention relates to a method wherein the polymerisable compound is represented by formula I in which
G is X,
each X represents a moiety comprising hydroxyethylacrylate or hydroxyethylmethacrylate,
each Y represents 0,
each R represents hydrogen,
L represents an amino acid moiety,
n=1,
W is O,
Q is O,
Z is a methyl, ethyl or n-propyl group.

The bioactive moiety is for example selected from pharmaceuticals, stabilisers, antithrombotic moieties, moieties increasing hydrophilicity or moieties increasing hydrophobicity.

The bioactive moiety may for instance be selected from cell signalling moieties, moieties capable of improving cell adhesion to the compound, polymer or article, moieties capable of controlling cell growth (such as stimulation or suppression of proliferation), anti-thrombotic moieties, moieties capable of improving wound healing, moieties capable of influencing the nervous system, moieties having selective affinity for specific tissue or cell types and antimicrobial moieties. The moiety may exert an activity when bound to the remainder of the compound, polymer or article and/or upon release therefrom. Examples of bioactive moieties that may be conjugated include perfluoroalkanes, polyalkylene oxides, such as polyethylene oxide and polypropylene oxide (increasing hydrophilicity and/or for reduced fouling); polyoxazolines; amino acids; peptides, including cyclic peptides, oligopeptides, polypeptides, glycopeptides and proteins, including glycoproteins; nucleotides, including mononucleotides, oligonucleotides and polynucleotides; and carbohydrates. Preferably amino acids, peptides or proteins are conjugated.

An amino acid may be conjugated for stimulating wound healing (arginine, glutamine) or to modulate the functioning of the nervous system (asparagine).

Peptides can be epitopes which may enhance or suppress biological response for example cellular growth proliferation or enhanced cell adhesion. In the case that for example enhanced antibody binding is required epitopes are the most obvious choice.

Examples of peptides comprise the sequences as given in table I, which are composed of amino acids, the abbreviations of which are known by a man skilled in the art.

TABLE I

| Peptide | suggested function |
|---|---|
| RGD, GRGDS, RGDS | Enhance bone and/or cartilage tissue |
| | formation; Regulate neurite outgrowth; |
| | Promote myoblast adhesion, |
| | proliferation and/or differentiation; |
| | Enhance endothelial cell adhesion |
| | and/or proliferation |
| PHSRN | Synergistic peptide for RGD |
| KQAGDV | Smooth muscle cell adhesion |
| YIGSR | Cell adhesion |
| REDV | Endothelial cell adhesion |
| GTPGPQGIAGQRGVV (P-15) | Cell adhesion (osteoblasts) |
| PDGEA | Cell adhesion (osteoblasts) |
| IKVAV | Neurite extension |
| RNIAEIIKDI | Neurite extension |
| KHIFSDDSSE | Astrocyte adhesion |
| VPGIG | Enhance elastic modulus of artificial |
| | extra- cellular-matrix (ECM) |
| FHRRIKA | Improve osteoblastic mineralization |
| KRSR | Osteoblast adhesion |
| KFAKLAARLYRKA | Enhance neurite extension |
| KHKGRDVILKKDVR | Enhance neurite extension |
| YKKIIKKL | Enhance neurite extension |
| NSPVNSKIPKACCVPTELSAI | Osteoinduction |
| APGL | Collagenase mediated degradation |
| VRN | Plasmin mediated degradation |
| AAAAAAAAA | Elastase mediated degradation |
| Ac-GCRDGPQ-GIWGQDRCG | Encourage cell-mediated proteolytic |
| | degradation, remodeling and/or |
| | bone regeneration (with RGD and |
| | BMP-2 presentation in vivo) |
| angiotensin | Vasoconstriction, increased blood |
| | pressure, release of aldosterone |
| | from the adrenal cortex. |
| HSWRHFHTLGGG | Binds to monocyte chemo attractant |
| | protein (MCP-1) |

A preferred example of a cyclic peptide is gramacidin S, which is an antimicrobial.

Further examples of suitable peptides in particular include: vascular endothelial growth factor (VEGF), transforming growth factor β (TGF-β), basic fibroblast growth factor (bFGF), epidermal growth factor (EGF), osteogenic protein (OP), monocyte chemoattractant protein (MCP 1), tumour necrosis factor (TNF), Examples of proteins which may in particular form part of a compound of the invention include growth factors, chemokines, cytokines, extracellular matrix proteins, glycosaminoglycans, angiopoetins, ephrins and antibodies.

A preferred carbohydrate is heparin, which is antithrombotic.

A nucleotide may in particular be selected from therapeutic nucleotides, such as nucleotides for gene therapy and nucleotides that are capable of binding to cellular or viral proteins, preferably with a high selectivity and/or affinity.

Preferred nucleotides include aptamers. Examples of both DNA and RNA based aptamers are mentioned in Nimjee et. al. Annu. Rev. Med. 2005, 56, 555-583. The RNA ligand TAR (Trans activation response), which binds to viral TAT proteins or cellular protein cyclin T1 to inhibit HIV replication, is an example of an aptamer. Further, preferred nucleotides include VA-RNA and transcription factor E2F, which regulates cellular proliferation.

The hydrolytic enzyme is preferably chosen from the group of carboxylic ester hydrolases (E.C. 3.1.1), thioester hydrolases (E.C.3.1.2) or peptidases (E.C. 3.4).

Preferably the hydrolytic enzyme is a peptidase selected from the group of serine-type carboxypeptidases (E.C. 3.4.16), metallocarboxypeptidases (E.C. 3.4.17), cysteine type carboxypeptidases (E.C. 3.4.18), serine endopeptidases (E.C. 3.4.21), cysteine endopeptidases (E.C. 3.4.22), aspartic endopeptidases (E.C. 3.4.23) or metallo endopeptidases (E.C. 3.4.24). Most preferred the enzyme is a serine endopeptidase such as subtilisin (E.C. 3.4.21.62), preferably subtilisin Carlsberg or a cysteine endopeptidase such as papain (E.C. 3.4.22.2). The enzyme may also be chosen from carboxylic ester hydrolases preferably selected from Candida antarctica lipase B (CALB), lypozyme RM, Piccantase A®, Rhizomucor miehei lipase, thermostable esterase or lilipase.

The hydrolytic enzyme may be obtained or derived from any organism, in particular from an animal, a plant, a bacterium, a mould, a yeast or a fungus. When referred to an enzyme from a particular source, recombinant enzymes originating from a first organism, but actually produced in a (genetically modified) second organism, are specifically meant to be included as enzymes from that first organism.

The hydrolytic enzymes may be immobilized, in particular loaded on a support such as, for example, an acrylic support, or used in their unsupported, i.e., free form. Suitable immobilisation techniques are generally known in the art.

In particular good results have been achieved with a peptidase, especially with an endopeptidase, more preferably with papain or subtilisin in order to conjugate a pendant carboxylic acid, ester or thioester, more in particular to conjugate a pendant methyl ester.

The amount of enzyme present or used in the process is difficult to determine in absolute terms (e.g. grams), as its purity is often low and a proportion may be in an inactive, or partially active, state. More relevant parameters are the activity of the enzyme preparation and the activities of any contaminating enzymes. These activities are usually measured in terms of the activity unit (U) which is defined as the amount which will catalyse the transformation of 1 micromole of the substrate per minute under standard conditions. Typically, this represents 10⁻⁶-10⁻¹¹ kg for pure enzymes and 10⁻⁴-10⁻⁷ kg for industrial enzyme preparations. The amount of hydrolytic enzyme per gram of polymer or polymerisable compound in principle is not critical and may for instance depend on the reactivity of the pendant carboxylic acid, ester or thioester group and on the enzyme cost price. A typical amount of enzyme ranges from 0.01-1000 Upper gram of polymer of polymerisable compound. Preferably 0.1-100 U/g are used and most preferably 1-10 U/g.

The conjugation of the bioactive moiety to the polymer or polymerisable compound can in general be carried out under mild and/or environmentally friendly conditions. For instance, no highly acidic or alkaline conditions are required which would hydrolyse any hydrolysable groups present in the polymer or polymerisable compound. Usually, the conjugation may be carried out at an approximately neutral pH, a slightly alkaline or a slightly acidic pH, for example at a pH between 4-10. The particular pH, which depends on the polymer or a polymerisable compound, the enzyme and the reaction conditions can easily be determined by the man skilled in the art.

In principle also a more alkaline or acidic pH may be used, in particular if the enzyme shows sufficiently selective activity. A favorable pH may be chosen based on a known or empirically determinable activity curve for the enzyme as a function of pH and the information disclosed herein.

The method in accordance with the invention may be carried out in water, in a mixture of water and one or more water-miscible organic solvent(s), in a mixture of water and one or more water-immiscible organic solvent(s) or in one or more organic solvent(s). In case that one or more organic solvent(s) is/are used it may be selected from the group of lower alcohols, for example methanol, ethanol, propanol, butanol, pentanol and hexanol. The alcohol may be a primary, secondary or tertiary alcohol. Particularly preferred are tertiary alcohols, such as t-butanol or t-amylalcohol. The organic solvent may also be selected from acetonitrile, dimethylformamide (DMF), toluene, dioxane, acetone, ethylacetate, methyl-tert-butylether (MBTE).

The water content is dependant on the polymer or polymerisable compound, the enzyme and the reaction conditions.

The temperature of the enzymatic conjugation reaction can usually be chosen within wide limits, taken into account factors such as the activity of the enzyme as a function of temperature and the stability of the enzyme at a specific temperature. Usually, the temperature is at least 0° C., in particular at least 10° C., more preferably at least 15° C. Usually, the temperature is up to 80° C. more preferably up to 60° C.

The conjugation of the bioactive moieties may occur prior to, during or after polymerization in case of a polymerisable compound. The conjugation may even occur after the polymer is given a form. The form may for example be a coating, a film, porous scaffolds, micelles, microspheres, nanoparticles, liposomes, fibres, gels, rods or polymerosomes.

Polymers conjugated with bioactive moieties are widely used not only in the pharmaceutical sector where polymer-drug conjugates are used in chemotherapy and for controlled and targeted drug delivery with biologics but also in the use of polymer—peptide or antibody conjugates for targeted drug delivery. Furthermore polymer—peptide conjugates are also used as materials for tissue engineering.

The invention will now be illustrated by the following examples without being limited thereto.

### Methods and Materials

### HPLC Method

Analytical HPLC diagrams were recorded on an HP1090 Liquid Chromatograph, using an Inertsil ODS-3 (150 mm length, 4.6 mm internal diameter) column at 40° C. UV detection was performed at 220 nm using a UVVIS 204 Linear spectrometer. The gradient program was: 0-25 min linear gradient ramp from 5% to 98% buffer B and from 25.1-30 min to 5% buffer B (buffer A: 0.5 ml/L methane sulfonic acid (MSA) in H₂O, buffer B: 0.5 ml/L MSA in acetonitrile). The flow was 1 mL/min from 0-25.1 min and 2 ml/min from 25.2-29.8 min, then back to 1 ml/min until stop at 30 min. Injection volumes were 20 μL. HPLC-MS diagrams were recorded on an Agilent 1100 series system using the same column and identical flow conditions as for analytical HPLC.

### Retention Times:

LDI-(HEMA)₂-OMe: 17.02 min,
LDI-(HEMA)₂-OH: 15.10 min
LDI-(HEMA)-₂-Gly-NH₂: 13.56 min
LDI-(HEMA)-₂-Gly-Gly: 13.61 min
LDI-(HEMA)-₂-Gly-Phe: 16.24 min
LDI-(HEMA)-₂-Gly-Phe-NH₂: 15.70 min
LDI-(HEMA)-₂-Ser-Trp: 15.46 min
LDI-(HEMA)-₂-Gly-Arg: 10.41 min
LDI-(HEMA)-₂-Gly-Ala-Gly: 13.15 min
LDI-(HEMA)₂Gly-Arg-Gly-Asp-Ser: 9.81 min
LDI-(HEMA)-₂-Gly-Arg-(Pmc)-Gly-Asp-(O^{t}Bu)-Ser-(O^{t}Bu)₂: 23.82 min
LDI-(HEMA)-₂-Leu-NH₂: 15.7 min
LDI-(HEMA)-₂-Leu-O^{t}Bu: 21.5 min
LDI-(HEMA)-₂-Val-NH₂: 14.8 min
LDI-(HEMA)-₂-Leu-Phe: 18.3 min
LDI-(HEMA)-₂-Leu-Pro-Pro: 15.9 min
LDI-(HEMA)₂-Ile-Pro-Pro: 15.8 min
LDI-(4-pentene)₂-OMe: 20.5 min
LDI-(4-pentene)₂-OH, 17.4 min
LDI-(4-pentene)-₂-Gly-Arg-Gly-Asp-Ser: 10.9 min
LDI-(4-pentene)-₂-Gly-Arg-(Pmc)-Gly-Asp-(O^{t}Bu)-Ser-(O^{t}Bu)₂: 25.0 min
LDI-(4-pentene)-₂-Leu-Leu-O^{t}Bu: 23.8 min
LDI-(4-pentene)-₂-Leu-Pro-Pro: 18.2 min
LDI-(4-pentene)-₂-Ser-Trp: 18.4 min
LDI-(4-pentene)-₂-Gly-NH₂: 14.74 min

### Materials

### I. Synthesis of LDI-(HEMA)₂-OMe (FIG. 1)

N^{α},N^{ε}-di-(2-methacryloxy-ethoxycarbonyl)-L-lysine methylester (LDI-(HEMA)₂-OMe) was prepared as follows;

2-Hydroxyethyl-methacrylate (HEMA, 502 mmol) was added dropwise to L-lysine-diisocyanate methylester (251 mmol), tin-(II)-ethylhexanoate (0.120 g) and Irganox 1035 (150 mg) under dry air at controlled temperature (<5° C.). The reaction mixture was stirred at 40° C. for 18 h. During this time, the IR NCO vibrational stretch at v=2260 cm⁻¹ had disappeared. The solvent was evaporated in vacuum to give the product as oil.

¹H-NMR (300 MHz, CDCl₃, 22° C., TMS): δ6.13-6.10 (m, 2H), 5.57 (q, J=1.5 Hz, 2H), 5.36 (d, J=8.0 Hz, 1H), 4.85 (bs, 1H), 4.35-4.27 (m, 9H), 3.73 (s, 3H), 3.16 (q, J=6.4 Hz, 2H), 1.93 (s, 6H), 1.88-1.76 (m, 1H), 1.74-1.61 (m, 1H), 1.55-1.44 (m, 2H), 1.42-1.30 (m, 2H).

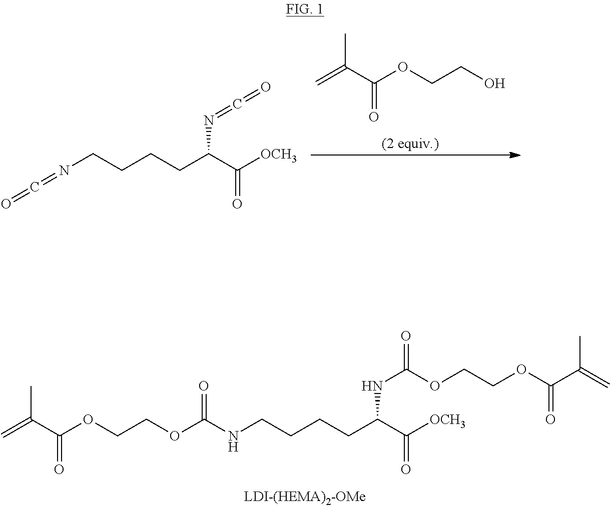

II. Synthesis of LDI-(4-pentene)₂-OMe (FIG. 2)

N^{α},N^{ε}-di-(4-penten-1-oxycarbonyl)-L-lysine methylester (LDI-(4-pentene)₂-OMe)

(FIG. 2) was prepared as follows;

L-Lysine-diisocyanate-methylester (5.3 g, 25 mmol) was dissolved in dry tetrahydrofuran (30 mL). To the resulting solution tin (II) 2-ethylhexanoate (25 mg, 0.061 mmol) was added and the solution was cooled to 0° C. and 4-pentenol (4.3 g, 50 mmol) was added dropwise over 30 minutes. The reaction was monitored by IR spectroscopy (2260 cm⁻¹, —N═C═O). After 2 h 37.5 mg of tin(II) 2-ethylhexanoate (0.092 mmol) was added. The reaction was kept at 0° C. for 1 additional h and then stirred for 18 h at room temperature. Finally, the organic solvent was removed in vacuum to obtain 9.6 g (25 mmol, 100% yield) of the title compound as colorless oil.

¹H-NMR (300 MHz, CDCl₃, 22° C., TMS): δ(ppm)=5.80-5.66 (m, 2H, —CH═CH₂); 5.16 (m, 1H, —NH—CH₂); 4.95-5.02 (m, 4H, —CH═CH₂); 4.62 (m, 1H, —CH—); 4.26 (m, 1H, —NHCH—); 4.0 (m, 4H, —(C═O)OCH₂—); 3.69 (s, 3H, —CH₃); 3.10 (m, 2H, —CH₂CH₂NH—); 2.05 (m, 4H, CH₂═CHCH₂—); 1.82-1.41 (m, 10H, CH₂═CHCH₂CH₂—, —NHCH₂CH₂CH₂CH₂—).

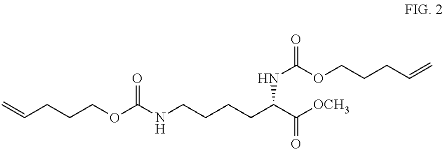

### EXAMPLE 1

### Peptide Coupling to LDI-(HEMA)₂-OMe and to LDI-(4-Pentene)₂-OMe by Subtilisin-A (FIG. 3)

To a stirred solution of 110 μmol LDI-(HEMA)₂-OMe or LDI-(4-pentene)₂-OMe in 1.5 mL of acetonitrile was added a solution of 2-4 equiv of amino acid or peptide derivative and 2-4 equiv of piperidine dissolved in 2.6 mL of DMF, as shown in tables II and III. Subsequently, 22 mg of Subtilisin-A (batch n. 8356056 activity 7-15 units per mg from Novozyme), dissolved in 0.2 mL of distilled H₂O was added and the reaction mixture was stirred at ambient temperature. The reaction was monitored by HPLC analysis.

Samples of 10 μL were withdrawn from the reaction mixture at regular time intervals. The 10 μL samples were diluted with 0.5 mL acetonitrile or methanol, filtered over a syringe filter (Agilent Technologies, membrane in regenerated cellulose, 0.45 μm pore size, 13 mm diameter) and analyzed by HPLC.

The product was identified by HPLC-MS using the non-purified reaction mixtures or by comparison of the HPLC diagram with the HPLC diagram of a chemically synthesized reference compound. HPLC-MS diagrams were recorded on an Agilent 1100 series system using the same column and identical flow conditions as for analytical HPLC. Results are given in tables II and III.

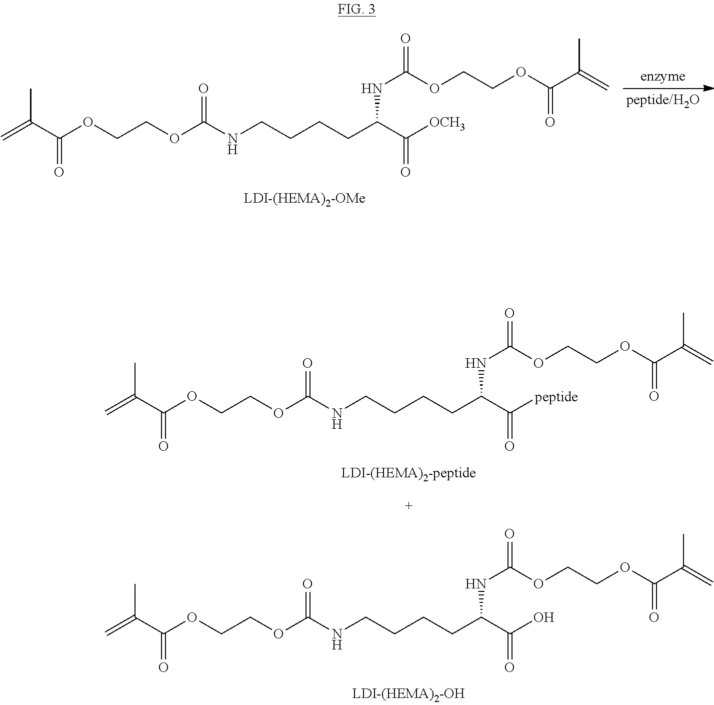

During the reaction, LDI-(HEMA)₂-OMe starting material is converted to the desired product LDI-(HEMA)-₂-peptide by enzymatic coupling with the peptide (or amino acid) nucleophile. Due to the hydrolytic activity of the selected enzyme, LDI-(HEMA)₂-OMe is partially hydrolysed to the corresponding LDI-(HEMA)₂-OH (if water is present).

In a typical final reaction mixture, the compounds present are: starting material LDI-(HEMA)₂-OMe, peptide (or amino acid), product LDI-(HEMA)-2-peptide (or LDI-(HEMA)-₂-amino acid) and hydrolysed LDI-(HEMA)₂-OH.

For the coupling of LDI-(4-pentene)₂-OMe the same reaction scheme holds.

### EXAMPLE 2

### Peptide Coupling to LDI-(HEMA)₂-OMe and to LDI-(4-pentene)₂-OMe by Papain

To a stirred solution of 110 μmol of LDI-(HEMA)₂-OMe or LDI-(4-pentene)₂-OMe in 1.2 mL of acetonitrile was added a solution of 2-8 equiv of amino acid or peptide derivative. In case the amino acid or peptide derivative was used as HCl salt the same equiv of triethylamine were added (see table II). Subsequently, 10 mg dithiothreitol (DTT), 100 mg of papain (from Merck, from Carica papaya, 30000USP-U/mg, art. 7144, batch n. 333 F677044,) and 0.8 mL of a 100 mM buffer as indicated in tables II and III were added and the reaction mixture was stirred at 37° C.

The reaction was monitored by HPLC analysis. Samples of 10 μL were withdrawn from the reaction mixture at regular time intervals. The 10 μL samples were diluted with 0.5 mL acetonitrile, filtered over a syringe filter (Agilent Technologies, membrane in regenerated cellulose, 0.45 μm pore size, 13 mm diameter) and analyzed by HPLC.

The product was identified by HPLC-MS using the non-purified reaction mixtures or by comparison of the HPLC diagram with the HPLC diagram of a chemically synthesized reference compound. HPLC-MS diagrams were recorded on an Agilent 1100 series system using the same column and identical flow conditions as for analytical HPLC.

Results are given in tables II and III.

TABLE II
Reactions using LDI-(HEMA)₂-OMe as starting material

| Amino acid or | | | | | Product | |
|---|---|---|---|---|---|---|
| peptide (and | | | | Reaction | Area % | HPLC- |
| triethylamine | | Enzyme/organic | | time | HPLC | MS con- |
| (TEA)), if added. | Equiv. | solvent(s)/buffer | pH | h | % | firmed |
| Gly-NH₂ | 4 | Sub/DMF/CH₃CN/H₂O | nd | 4.5 | 87^{#} | yes |
| Gly-Gly | 4 | Sub/DMF/CH₃CN/H₂O | nd | 4.5 | 69^{#} | yes |
| Gly-Phe | 4 | Sub/DMF/CH₃CN/H₂O | nd | 4.5 | 60^{#} | yes |
| Gly-PheNH₂ | 4 | Sub/DMF/CH₃CN/H₂O | nd | 4.5 | 81^{#} | yes |
| Ser-Trp | 4 | Sub/DMF/CH₃CN/H₂O | nd | 4.5 | 27^{#} | yes |
| Gly-Arg | 4 | Sub/DMF/CH₃CN/H₂O | nd | 4.5 | 32^{#} | no |
| Gly-Ala-Gly | 4 | Sub/DMF/CH₃CN/H₂O | nd | 5 | 35^{#} | no |
| Gly-Arg-Gly-Asp- | 2 | Sub/DMF/CH₃CN/H₂O | nd | 4 | 19^{#} | yes |
| Ser | | | | | | |
| Gly-Arg-(PMC)-Gly- | 2 | Sub/DMF/CH₃CN/H₂O | nd | 4 | 5* | yes |
| Asp-(O^{t}Bu)-Ser- | | | | | | |
| (O^{t}Bu)₂ | | | | | | |
| Leu-NH₂ | 8 | Pap/CH₃CN/McIlvaine, | 6 | 3.5 | 60^{#} | yes |
| Leu-NH₂ | 8 | Pap/CH₃CN/ | 8 | 3.5 | 53^{#} | no |
| | | Triethanolamine | | | | |
| Leu-O^{t}Bu•HCl | 8 | Pap/CH₃CN/ | 8 | 3.5 | 26^{#} | no |
| | | Triethanolamine | | | | |
| Leu-O^{t}Bu•HCl + TEA | 8 | Pap/CH₃CN/ | 8 | 3.5 | 42^{#} | yes |
| | | Triethanolamine | | | | |
| Val-NH₂•HCl + TEA | 8 | Pap/CH₃CN/McIlvaine | 6 | 4 | 39^{#} | no |
| Leu-Phe | 8 | Pap/CH₃CN/ | 8 | 4 | 40^{#} | no |
| | | Triethanolamine | | | | |
| Ser-Trp | 2 | Pap/CH₃CN/Phosphate | 9 | 2 | 27^{#} | no |
| Leu-Pro-Pro | 2 | Pap/CH₃CN/Phosphate | 9 | 4 | 73^{#} | no |
| Ile-Pro-Pro | 1 | Pap/CH₃CN/Phosphate | 9 | 4 | 61^{#} | no |

*Sub = Subtilisin; Pap = Papain
*nd = non determined

The reaction yield was determined by HPLC analysis, as area percentage, defined as follows:

$$Area \% = \frac{LDI \text{-} {( HEMA )}_{2} \text{-} peptide}{( \begin{matrix}LDI \text{-} {( HEMA )}_{2} \text{-} O Me + LDI \text{-} {( HEMA )}_{2} \text{-} O H + \\ LDI \text{-} {( HEMA )}_{2} \text{-} peptide\end{matrix} )} \times 100$$

In the case the peptide or amino acid contains a Pmc group; the reaction yield was determined by HPLC analysis as area percentage, defined as follows:

$$Area \% = \frac{LDI \text{-} {( HEMA )}_{2} \text{-} peptide}{( peptide + LDI \text{-} {( HEMA )}_{2} \text{-} peptide )} \times 100$$

The reaction time as set in tables II and III correlates with the maximum conversion to the desired product.

TABLE III
Reactions using LDI-(4-pentene)₂-OMe as starting material

| | | | | | Product | |
|---|---|---|---|---|---|---|
| Amino acid or | | | | Reaction | Area % | HPLC- |
| peptide (and | | Enzyme/organic | | time | HPLC | MS con- |
| TEA if added) | Equiv. | solvent(s)/buffer | pH | h | % | firmed |
| Gly-Arg-Gly-Asp- | 2 | Sub/DMF/CH₃CN/H₂O | nd | 4 | 85* | yes |
| Ser | | | | | | |
| Gly-Arg-(Pmc)- | 2 | Sub/DMF/CH₃CN/H₂O | nd | 4 | 67^{#} | yes |
| Gly-Asp-(O^{t}Bu)- | | | | | | |
| Ser-(O^{t}Bu)₂ | | | | | | |
| Leu-O^{t}Bu•HCl | 2 | Pap/CH₃CN/Phosphate | 9 | 2 | 72^{#} | no |
| Leu-Pro-Pro | 2 | Pap/CH₃CN/Phosphate | 9 | 2 | 100^{#} | yes |
| Ser-Trp | 2 | Pap/CH₃CN/Phosphate | 9 | 2 | 5^{#} | no |

The reaction yield was determined by HPLC analysis, as area percentage, defined as follows:

$$Area \% = \frac{LDI \text{-} {( 4 \text{-} pentene )}_{2} \text{-} peptide}{( \begin{matrix}LDI \text{-} {( 4 \text{-} pentene )}_{2} \text{-} O Me + LDI \text{-} {( 4 \text{-} pentene )}_{2} \text{-} O H + \\ LDI \text{-} {( 4 \text{-} pentene )}_{2} \text{-} peptide\end{matrix} )} \times 100$$

In the case the peptide or amino acid contains a Pmc group, the reaction yield was determined by HPLC analysis as area percentage, defined as follows:

$$Area \% = \frac{LDI \text{-} {( 4 \text{-} pentene )}_{2} \text{-} peptide}{( peptide + LDI \text{-} {( 4 \text{-} pentene )}_{2} \text{-} peptide )} \times 100$$

It is clear from Tables II and III, that if the amino acid or peptide nucleophile has a Gly on the N-terminus a subtilisin is preferably used. If another amino acid or peptide nucleophile is used on the N terminus, papain is preferably used.

### EXAMPLE 3

### Peptide Coupling to LDI-(4-Peptene)₂-OMe by Cal-B

To a stirred solution of 0.5 mmol LDI-(4-pentene)₂-OMe in 2.0 mL of acetonitrile was added 4 equiv of H-Gly-NH₂.HCl (220 mg) and 4 equiv of piperidine (0.20 mL). Subsequently, 220 mg of Cal-B (from Novozyme, lipase Novozym 435 from Candida Antarctica, batch n. LC200204) was added and the reaction mixture was stirred at 50° C. After 3 days 15% of the starting material had been converted to the LDI-(4-pentene)-₂-Gly-NH₂) product.

The product was identified by HPLC-MS using the non-purified reaction mixtures and by comparison of the HPLC diagram with the HPLC diagram of a chemically synthesized reference compound. HPLC-MS diagrams were recorded on an Agilent 1100 series system using the same column and identical flow conditions as for analytical HPLC.

Data are HPLC area percentage:

$$Area \% = \frac{LDI \text{-} {( 4 \text{-} pentene )}_{2} \text{-} Gly \text{-} {NH}_{2}}{( \begin{matrix}LDI \text{-} {( 4 \text{-} pentene )}_{2} \text{-} O Me + LDI \text{-} {( 4 \text{-} pentene )}_{2} \text{-} O H + \\ LDI \text{-} {( 4 \text{-} pentene )}_{2} \text{-} Gly \text{-} {NH}_{2}\end{matrix} )} \times 100$$

1. Method for the selective conjugation of bioactive moieties to a pendant carboxylic acid, ester or thioester group in which the pendant group is part of a polymer or a polymerisable compound, wherein the method comprises contacting the polymer or polymerisable compound with a hydrolytic enzyme to catalyse the conjugation between the bioactive moiety and the pendant carboxylic acid, ester or thioester group.

2. Method according to claim 1, wherein the pendant carboxylic acid, ester or thioester group is part of a polymer or a polymerisable compound comprising (a) at least two polymerisable moieties and (b) at least one amino acid residue.

3. Method according to claim 1 wherein the polymer or polymerisable compound comprises—in addition to the pendant carboxylic acid, ester or thioester group, a moiety selected from urea groups, thio-urea groups, urethane groups, thio-urethane groups, ester groups, amide groups, glycopeptide groups, carbonate groups, sulphones or carbohydrate groups.

4. Method according to claim 1 wherein the polymerisable
compound is represented by the formula I 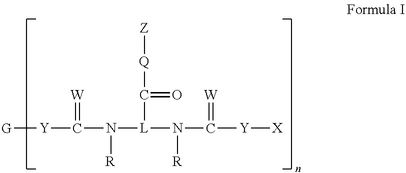
wherein
G is a residue of a polyfunctional compound having at least n functional groups or a moiety X;
each X independently represents a moiety comprising a polymerisable group;
each Y independently represents O, S or NR;
each R independently represents hydrogen or a group selected from substituted and unsubstituted hydrocarbons which optionally contain one or more heteroatoms;
L represents a substituted or unsubstituted hydrocarbon which optionally contains one or more heteroatoms;
n is an integer having a value of at least 1;
W is O or S;
Q is O or S;
Z is H or a substituted or unsubstituted hydrocarbon group.

5. Method according to claim 4 whereby
G is X;
each Y═O and each X represents a moiety comprising hydroxyalkylene, hydroxyethylacrylate or hydroxyethyl methacrylate;
each R represents hydrogen;
L represents an amino acid moiety;
n=1;
W is O;
Q is O;
Z is H or an alkyl group with 1-6 C atoms

6. Method according to claim 5 whereby the amino acid moiety is chosen from a lysine moiety, a diaminopropionic acid moiety, a hydroxyllysine moiety, a N-alpha-methylated lysine moiety or a diaminobutanoic acid moiety.

7. Method according to claim 5 whereby the amino acid residue has the L-configuration.

8. Method according to claim 1 wherein the polymer is a polymer composed of a compound.

9. Method according to claim 1 whereby the polymer or polymerisable compound contains one or more lysine-methylester moieties.

10. Method according to claim 1 wherein the hydrolytic enzyme is chosen from the group of carboxylic ester hydrolases, thioester hydrolases, or peptidases.

11. Method according to claim 1 wherein the hydrolytic enzyme is a peptidase selected from the group of serine-type carboxypeptidases, metal locarboxypeptidases, cysteine-type carboxypeptidases, serine endopeptidases, cysteine endopeptidases, aspartic endopeptidases and metalloendopeptidases.

12. Method according to claim 1 wherein the enzyme is a serine endopeptidase.

13. Method according to claim 12 wherein the enzyme is subtilisin.

14. Method according to claim 13 wherein the enzyme is subtilisin Carlsberg.

15. Method according to claim 1 wherein the enzyme is a cysteine endopeptidase.

16. Method according to claim 15 wherein the enzyme is papain.

17. Method according to claim 10 wherein the enzyme is a carboxylic ester hydrolase selected from Candida antarctica lipase B (CALB), lypozyme RM, Piccantase A®, Rhizomucor miehei lipase, thermostable esterase or lilipase.

18. Method according to claim 1 whereby the conjugation of the bioactive moieties may occur prior to, during or after polymerization of the polymerisable compound.

19. Method according to claim 1 whereby the conjugation of the bioactive moieties occurs after the polymer is given a form.

20. Method according to claim 1 whereby the bioactive moiety is chosen from amino acids, peptides or proteins.
